(19) United States
(12) Patent Application Publication
(10) Pub. No.: US 2004/0219663 A1
(43) Pub. Date: Nov. 4, 2004

(54) Biopolymer array fabrication using different drop deposition heads

(76) Inventors: Robert D. Page, San Jose, CA (US); Eric M. Leproust, Campbell, CA (US); John P. Scalia, San Mateo, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/427,764

(22) Filed: Apr. 30, 2003

(51) Int. Cl. C12M 1/34; B05D 3/00; B41J 2/145; B41J 2/15

(52) U.S. Cl. 435/287.2; 427/2.11; 347/40

(57) ABSTRACT

A method of fabricating an array of biopolymer probes bound to a surface of a substrate at feature locations. The method uses multiple deposition heads each with a set of nozzles through which drops are dispensed. Drops are deposited during a same cycle onto the feature location from a set of the deposition heads while spaced from the surface. At least some of those drops contain probe precursors, so that the probe precursors bind to the surface through a linker. This depositing is repeated multiple times with the probe precursor deposited in a prior cycle serving as the linker for a probe precursor deposited in a subsequent cycle. Additionally, drops are deposited at a same feature from the set of heads during a one cycle, and further drops are deposited at the same feature from the set of heads during another cycle. Apparatus and computer program products are also provided.

### FIELD OF THE INVENTION

This invention relates to arrays, particularly polynucleotide arrays such as DNA arrays, which are useful in diagnostic, screening, gene expression analysis, and other applications.

### BACKGROUND

Biopolymer arrays (such as polynucleotide arrays of DNA or RNA) are known and are used, for example, as diagnostic or screening tools. Such arrays include regions of usually different sequence biopolymers arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example in the case of polynucleotide arrays all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers onto a substrate, or by in situ synthesis methods. The in situ fabrication methods for synthesizing peptide or polynucleotide arrays include those described in U.S. Pat. No. 5,449,754, U.S. Pat. No. 6,180,351, U.S. Pat. No. 6,534,627 and WO 98/41531 and the references cited in each. The in situ method for fabricating a biopolymer array typically follows, at each of the multiple different addresses at which features are to be formed, the same conventional iterative sequence used in synthesizing the biopolymer on a support by means of known chemistry. In the case of polynucleotides a particular method uses a nucleoside reagent of the formula:

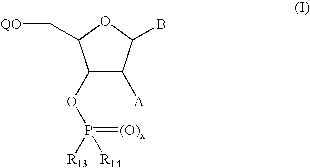

in which:

A represents H, alkyl, or another substituent which does not interfere in the coupling of compounds of formula (I) to form polynucleotides according to the in situ fabrication process;

B is a purine or pyrimidine base whose exocyclic amine functional group is optionally protected;

Q is a conventional protective group for the 5′—OH functional group;

x=0 or 1 provided:

a) when x=1:

R₁₃ represents H and R₁₄ represents a negatively charged oxygen atom; or

R₁₃ is an oxygen atom and R₁₄ represents either an oxygen atom or an oxygen atom carrying a protecting group; and

b) when x=0, R₁₃ is an oxygen atom carrying a protecting group and R₁₄ is either a hydrogen or a di-substituted amine group.

When x is equal to 1, R₁₃ is an oxygen atom and R₁₄ is an oxygen atom, the method is in this case the so-called phosphodiester method; when R₁₄ is an oxygen atom carrying a protecting group, the method is in this case the so-called phosphotriester method.

When x is equal to 1, R₁₃ is a hydrogen atom and R₁₄ is a negatively charged oxygen atom, the method is known as the H-phosphonate method.

When x is equal to 0, R₁₃ is an oxygen atom carrying a protecting group and R₁₄ is either a halogen, the method is known as the phosphite method and; when x =0, R₁₃ is an oxygen atom carrying a protecting group, and R₁₄ is a leaving group of the disubstituted amine type, then compounds of formula I are known as “nucleoside phosphoramidites” and the method is known as the phosphoramidite method.

The conventional sequence used to prepare an oligonucleotide using reagents of the type of formula (I), basically follows the following steps: (a) coupling a selected nucleoside through a phosphite linkage to a functionalized support in a first cycle, or a nucleoside bound to the substrate (i.e. the nucleoside-modified substrate) in subsequent cycles; (b) optionally blocking (“capping”) unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle in which these steps are repeated. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner. The nucleoside reagent in (a) generally requires activation by a suitable activator such as tetrazole.

Protecting groups which may be used include those such as described in “Protective groups in organic synthesis” by Theodora W. Greene and Peter G. M. Wuts, Wiley-interscience ISBN 0-471-62301-6 p.68-117, and may be made by methods described therein or otherwise.

The foregoing methods of preparing polynucleotides are described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere The phosphoramidite and phosphite triester approaches are most broadly used, but other approaches include the phosphodiester approach, the phosphotriester approach and the H-phosphonate approach.

In the case of array fabrication, different monomers may be deposited at different addresses on the substrate during any one cycle so that the different features of the completed array will have different desired biopolymer sequences. One or more intermediate further steps may be required in each cycle (that is, between depositions of different cycles), such as the conventional oxidation and washing steps. One particularly useful way of depositing monomers is by depositing drops each containing a monomer from a pulse jet spaced apart from the substrate surface, onto the substrate surface. Such techniques are described in detail in, for example, U.S. Pat. No. 6,242,266, U.S. Pat. No. 6,232,072, U.S. Pat. No. 6,180,351, and U.S. Pat. No. 6,171,797. In one prior art method for fabricating DNA arrays two different deposition heads were used, each of which deposited two of the four phosphoramidites and a tetrazole activator. As each head passed over the substrate in a cycle it would deposit a phosphoramidite onto the substrate and the same head would deposit the tetrazole activator. This would be repeated in subsequent cycles.

In array fabrication, the probes formed at each feature are usually expensive. Additionally, sample quantities available for testing are usually also very small and it is therefore desirable to simultaneously test the same sample against a large number of different probes on an array. These conditions make it desirable to produce arrays with large numbers of very small, closely spaced features. To facilitate correct interpretation of the data from such arrays, it is important that the features have the characteristics of actually being present at the expected location, and that the different features are of a homogeneous composition. Features which are non-homogeneous (for example, by having portions carrying primarily incorrect probe sequences) may unexpectedly bind to components in a sample to which the array is exposed, leading to mis-interpretation of results.

It is desirable then to provide an array fabrication process which can provide features of good homogeneity in their composition.

### SUMMARY OF THE INVENTION

The present invention then, provides a method of fabricating an array of biopolymer probes bound to a surface of a substrate at feature locations. The method uses multiple deposition heads each with a set of nozzles through which drops are dispensed. In the method drops are deposited during a same cycle onto the feature location from a set of the deposition heads while spaced from the surface. At least some of those drops contain probe precursors, so that the probe precursors bind to the surface through a linker. This depositing is repeated multiple times with the probe precursor deposited in a prior cycle serving as the linker for a probe precursor deposited in a subsequent cycle. Additionally, drops are deposited at a same feature from the set of heads during a one cycle, and further drops are deposited at the same feature from the set of heads during another cycle.

The set of nozzles in each head may be fixed nozzles. That is, they cannot be moved relative to each other but are all moved when the head is moved. In another aspect of the invention the nozzles may not be fixed. In this aspect the method may include adjusting the relative orientation of heads in the set so as to simultaneously adjust the trajectories of drops ejected from a set of nozzles of one head relative to a set of nozzles of another head

The invention further provides computer program products and apparatus which can execute any one or more methods of the present invention. Such a computer program product may include a computer readable storage medium having a computer program stored on it which executes a method of the present invention. Such an apparatus may include multiple deposition heads of a type described herein, and a processor which controls operation of the deposition heads so as to execute a method of the present invention.

While the above specifically references biopolymers, it will be understood throughout this application that any desired polymer could be fabricated by methods of the present invention and accordingly, “biopolymer” can generally be replaced by polymer in the description herein (except with regard to the definition of “biopolymer” and the like).

Different various aspects of the present invention can provide any one or more of the following or other useful benefits in biopolymer array fabrication. For example, regions of features carrying incomplete biopolymer sequences may be reduced. In other aspects an aspect ratio of features can be controlled.

### BRIEF DESCRIPTION OF THE DRAWINGS

Embodiments of the invention will now be described with reference to the following drawings in which:

FIG. 1 illustrates a substrate carrying multiple arrays, such as may be fabricated by methods of the present invention;

FIG. 2 is an enlarged view of a portion of FIG. 1 showing multiple spots or features of one array; and

FIG. 3 is an enlarged illustration of a portion of the substrate of FIG. 1;

FIG. 4 shows a nozzle distribution in a two head system as may be used in a prior art apparatus or an apparatus of the present invention;

FIG. 5 illustrates deposited drops in two cycles from the head system of FIG. 4 where the prior art method of depositing a probe precursor and activator from the same head is used;

FIG. 6 illustrates deposited drops in two cycles from the head system of FIG. 4 where a method of the present invention is used to deposit a probe precursor from one head and an activator from another head;

FIG. 7 is similar to FIG. 6 but illustrates the result when all the drops deposited at the same feature location from the different heads during a same cycle do not together cover an area on the surface which is substantially coextensive over different cycles;

FIG. 8 illustrates deposited drops in two cycles from the head system of FIG. 4 where another method of the present invention is used in which a probe precursor is deposited from one head and an activator from both heads;

FIG. 9 is similar to FIG. 6 but illustrates two cycles of a method of the present invention in each of which a series of drops of the probe precursor are deposited from the one head and a series of drops of an activator from the other head;

FIG. 10 is similar to FIG. 9 but illustrates how a method of the present invention can be used to control an aspect ratio of an array feature;

FIG. 11 is a schematic diagram of an apparatus which can execute a method of the present invention; and

FIG. 12 is a flowchart illustrating a method of the present invention.

To facilitate understanding, identical reference numerals have been used, where practical, to designate the same elements which are common to different figures. Drawings are not necessarily to scale. Throughout this application any different members of a generic class may have the same reference number followed by different letters (for example, arrays 12a, 12b, 12c, and 12d may generically be referenced as “arrays 12”)

### DETAILED DESCRIPTION OF EMBODIMENTS OF THE INVENTION

Throughout the present application, unless a contrary intention appears, the following terms refer to the indicated characteristics.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes DNA (including cDNA), RNA and oligonucleotides, regardless of the source.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

A “nucleoside” is the same as a nucleotide but without the phosphate group present (for example, there may instead be a phosphite present).

An “oligonucleotide” generally refers to a nucleotide multimer of about 10 to 100 nucleotides in length, while a “polynucleotide” includes a nucleotide multimer having any number of nucleotides.

An “array”, unless a contrary intention appears, includes any one, two or three-dimensional arrangement of addressable regions bearing a particular chemical moiety or moieties (for example, biopolymers such as polynucleotide sequences) associated with that region. Each region may extend into a third dimension in the case where the substrate is porous while not having any substantial third dimension measurement (thickness) in the case where the substrate is non-porous. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). An array feature is generally homogenous and the features in the present invention will be separated by some intervening space. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other).

An “array layout” or “array characteristics”, refers to one or more physical, chemical or biological characteristics of the array, such as feature positioning, one or more feature dimensions, or some indication of an identity or function (for example, chemical or biological) of a moiety at a given location, or how the array should be handled (for example, conditions under which the array is exposed to a sample, or array reading specifications or controls following sample exposure).

“Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless).

An array “assembly” may be the array plus only a substrate on which the array is deposited, although the assembly may be in the form of a package which includes other features (such as a housing with a chamber). A “chamber” references an enclosed volume (although a chamber may be accessible through one or more ports).

It will also be appreciated that throughout the present application, that words such as “front”, “back”, “top”, “upper”, and “lower” are used in a relative sense only.

A “pulse jet” is any device which can dispense drops in the formation of an array. Pulse jets operate by delivering a pulse of pressure to liquid adjacent an outlet or orifice such that a drop will be dispensed therefrom (for example, from a piezoelectric or thermoelectric element in a same chamber as the orifice).

When drops are deposited during a same cycle and same feature location “onto a feature location”, “for a feature location” or “at a feature location”, or similar, this means that the drops overlap in their positions to some extent so that the feature can be formed. Thus, drops deposited onto a feature location during a same cycle contact one another at least partially such that their contents can mix by diffusion or otherwise. However, between cycles the array (and hence feature locations) will be washed of any unlinked precursors and other reagents and dried between so that drops from one cycle do not merge with drops from another cycle at the same feature (although the contents of later deposited drops, to the extent they overly linked precursors from a previous cycle, may react with overlaid linked precursors).

A “group” in relation to a chemical formula, includes both substituted and unsubstituted forms of the group where any substituents do not interfere with the desired reactions.

A “phospho” group includes a phosphodiester, phosphotriester, and H-phosphonate groups as defined in connection with formula (I) above, while a “phosphite” includes a phosphoramidite.

A “phosphoramidite” is a compound of formula (I) when x=0 and R₁₄ is a leaving group of the disubstituted amine type. A “nucleoside phosphoramidite” is a nucleoside having a phosphoramidite group such as at the 3′ or 5′ position on the furyl ring in formula (I). A particular example is a nucleoside phosphoramidite of formula (I) above in which A is H; x=0; R₁₃ is a protected oxygen in the form of —OCH₂CH₂CN; R₁₄ is —N(isopropyl)₂; the protecting group Q is trityl; and B is any of the usual four protected purine or pyrimidine bases of nucleosides

“Lower alkyl group” or other “lower” group references either such group with from 1 to 6 C atoms (such as 2, 3, 4, or 5 C atoms).

A “blocked” hydroxy group references a hydroxy group (—OH) in which the free H has been replaced by a protecting group which renders the hydroxy unreactive under the conditions of an in situ biopolymer fabrication process in which it is used.

“Fluid” is used herein to reference a liquid.

“May” refers to optionally.

Any recited method can be carried out in the order of events recited or in any other order which is logically possible. Reference to a singular item, includes the possibility that there are plural of the item present. All patents and other references cited in this application, are incorporated into this application by reference except insofar as anything in those patents or references, including definitions, conflicts with anything in the present application (in which case the present application is to prevail).

In particular embodiments of the present invention the biopolymer probes comprise polynucleotide or peptide probes. In such cases the precursors used may be polynucleotides or poly-amino acids of shorter length than the final sequence fabricated at a feature. More typically, the precursors are monomers (a single nucleoside or amino acid). In one method of the present invention the set of heads includes a first head and a second head. In this case the drops deposited at the same feature from the set of heads during the one cycle include a drop of a probe precursor from the first head and a drop of an activator from the second head. Additionally, the drops deposited at the same feature in the other cycle comprise a drop of a probe precursor (the same or different from that deposited during the one cycle) from the second head and a drop of the activator from the first head.

In another method using the first and second heads, the drops deposited at the same feature from the set of heads during the one cycle includes a series of drops of the probe precursor from the first head and a series of drops of an activator from the second head. In this method the drops deposited at the same feature in the other cycle comprise a series of drops of the probe precursor from the second head and a series of drops of the activator from the first head. Alternatively, or additionally, the drops deposited at the same feature from the set of heads during the one cycle may include a drop of a probe precursor from the first head and a drop of an activator from the first head and from the second head. In this configuration the drops deposited at the same feature in the other cycle may include a drop of a probe precursor from the second head and a drop of the activator from the first head and from the second head.

In methods of the present invention all the drops deposited at the same feature location from the different heads during a same cycle may together cover an area on the surface which is substantially coextensive over different cycles. In this regard “substantially coextensive” and the like means substantially covering a same region on the surface so that they overlap by at least 60% of their areas (and more typically by at least 70%, 80%, 90%, 95%, or 100%). All the drops deposited at the same feature location from a same head during a same cycle may together have a coverage area which is substantially constant across different heads and different cycles. However, this need not be the case. For example, activator deposited from the first and second heads at the same feature during each of the cycles may each have a coverage area which is greater than the probe precursor deposited during the same cycle. A “coverage area” and the like in this regard refers to area (in mm² or some other dimension) on the surface and unlike “coextensive”, does not imply any particular location. Areas of coverage are “substantially constant” if they are with at least 60% of one another (and more typically by at least 70%, 80%, 90%, 95%, or 100%).

When series of drops are used in a method of the present invention, such as mentioned above, the drops within each series at the same feature may be displaced relative to one another along a first direction while series from different heads are displaced relative to one another in a direction crosswise to the first direction. As will be described further below, such a method can allow an aspect ratio of a feature to be controlled.

In a typical operation of the method drops of an activator and probe precursor may be dispensed from the first and second heads, with different probe precursors being dispensed from the first and second heads (and the same activator may be dispensed from different heads). In the particular situation where the biopolymer probe is polynucleotides, an activator and a pair of nucleoside monomers may be dispensed from each of the first and second heads, with different pairs of nucleoside monomers being dispensed from the first and second heads. The nucleoside monomers may be nucleoside phosphoramidites.

Various constructions of the head are possible in which the nozzles are fixed relative to one another. For example, the nozzles of the head may be formed in a one-piece orifice member. By nozzles in a head being “fixed” relative to one another is meant that they cannot be reversibly adjusted multiple times relative to one another without effectively rendering the nozzles useless. For example, nozzles in a one-piece nozzle plate cannot be reversibly adjusted relative to one another without re-drilling the holes which define the nozzles (and drilling cannot be reversed).

The deposited drops in any method of the present invention may include probe precursors or another reagent such as an activator (for example, tetrazole) to facilitate the linking of one probe precursor to a previously deposited probe precursor. The concentrations of the probe precursors in the drops may be varied as desired and may be less than 440, 420, 400, 380, 360, or 340 mM, or even less than 300, 250, 200, or 150 mM. The concentration of the activator in the drops may be varied as desired and maybe less than 1500, 1300, 1100, 900, 800, 600 mM, or even less than 400, 300, 200, or 100 mM.

Various solvents may be used in the drops, such as any of those described in U.S. Pat. No. 6,028,189, U.S. Pat. No. 6,384,210, and U.S. Pat. No. 6,419,883. Particular solvents may include an alkylene carbonate solvent, such as propylene carbonate. The biopolymer probes may particularly be polynucleotides and the probe precursors are nucleoside phosphoramidites.

Probe precursors may be any biomonomer, such as a nucleoside monomer (for example, a nucleoside phosphoramidite) or amino acid monomer, which has first and second linking groups such that a polynucleotide or peptide probe can be formed by a method which includes sequential deposit of different probe precursors onto a surface in different cycles. Such methods and suitable linking groups are described above and in the cited references, in relation to the in situ fabrication methods which, as mentioned above, are incorporated herein by reference particularly with regard to synthesis chemistry.

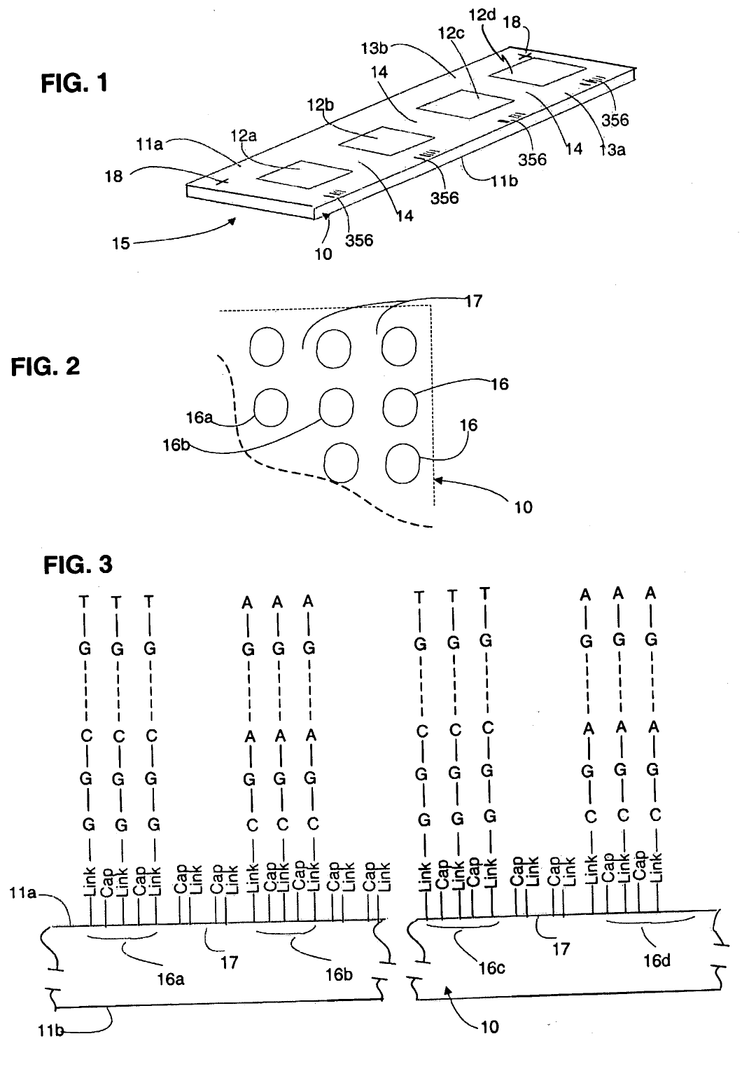

Referring now to FIGS. 1-3, an array assembly 15 (which may be referenced also as an “array unit”) fabricated by a method of the present invention may include a porous or non-porous substrate 10 which may be smooth or substantially planar, or have irregularities, such as depressions or elevations (although irregular substrate surfaces may make reading of the exposed array more difficult). Substrate 10 may also be in the form of a rigid substrate 10 (for example, a transparent non-porous material such as glass or silica) of limited length, carrying one or more arrays 12 disposed along a front surface 11a of substrate 10 and separated by inter-array areas 14. Alternatively, substrate 10 can be flexible (such as a flexible web). The substrate may be of one material or of multi-layer construction. A back side 11b of substrate 10 generally does not carry any arrays 12. The arrays on substrate 10 can be designed for testing against any type of sample, whether: a trial sample; reference sample; a combination of the foregoing; or a known mixture of polynucleotides, proteins, polysaccharides and the like (in which case the arrays may be composed of features carrying unknown sequences to be evaluated). While four arrays 12 are shown in FIG. 1, it will be understood that substrate 10 and the embodiments to be used with it, may use any number of desired arrays 12 such as at least one, two, five, ten, twenty, fifty, or one hundred (or even at least five hundred, one thousand, or at least three thousand). When more than one array 12 is present they may be arranged end to end along the lengthwise direction of substrate 10. Depending upon intended use, any or all of arrays 12 may be the same or different from one another and each will contain multiple spots or features 16 of biopolymers in the form of polynucleotides.

A typical array 12 may contain from more than ten, more than one hundred, more than one thousand or ten thousand features, or even more than from one hundred thousand features. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature of the same composition are excluded, the remaining features may account for at least 5%, 10%, or 20% of the total number of features).

In any aspect of the present invention, the features 16 may be spaced apart by a distance greater than 0 and less than 70%, 60% 50%, 25%, or 10% of a maximum dimension of the feature. Further, the features may have a maximum dimension of between 20 (or 50) to 100 (or 80) microns and are spaced apart by less than 130 microns (or by less than 100 or 50 microns). Various feature densities on the substrate surface are possible. For example, features having a maximum dimension greater than any of the foregoing figures may be present on the surface of at least 30 features/mm², 40 features/mm², or 60 features/mm². While round features 16 are shown, various other feature shapes are possible (such as elliptical). The features 16 may also be arranged in other configurations (for example, circular) rather than the rectilinear grid illustrated. Similarly, arrays 12 on a same substrate 10 need not be laid out in a linear configuration.

Each array 12 may cover an area of less than 100 cm², or even less than 50 cm², 10 cm² or 1 cm². In many embodiments, particularly when substrate 10 is rigid, it may be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 mm and less than 1.2 mm. When substrate 10 is flexible, it may be of various lengths including at least 1 m, at least 2 m, or at least 5 m (or even at least 10 m). With arrays that are read by detecting fluorescence, the substrate 10 may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

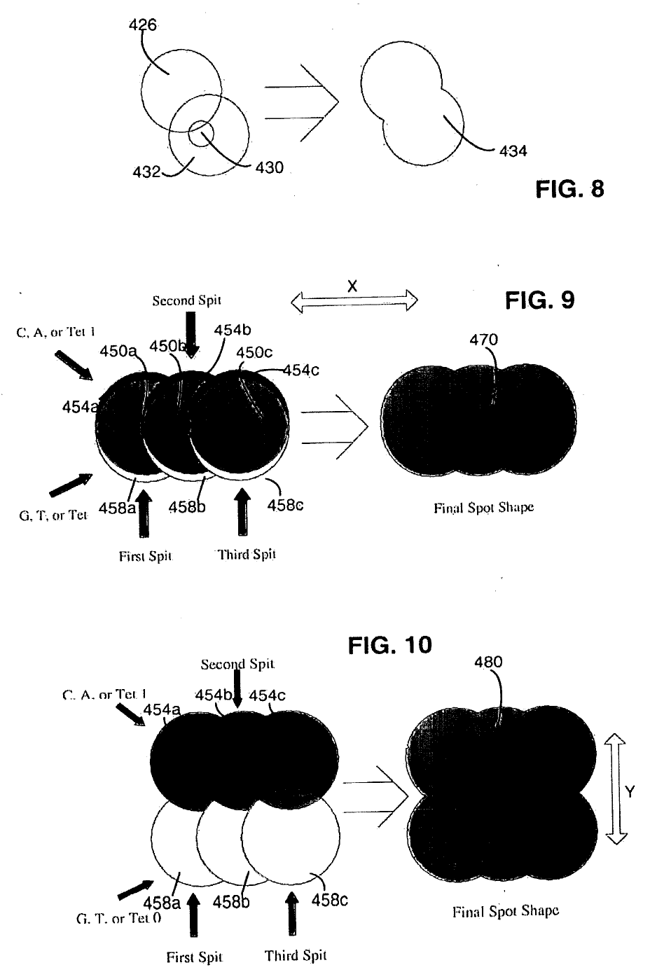

In the case where arrays 12 are formed by the in situ fabrication processes described above, interfeature areas 17 will typically be present which do not carry any polynucleotide. It will be appreciated though, that the interfeature areas 17 could be of various sizes and configurations. Each feature 16 carries a predetermined polynucleotide (which includes the possibility of mixtures of polynucleotides). As per usual, A, C, G, T represent the usual four nucleotides. “Link” (see FIG. 8 in particular) represents a linking agent (molecule) covalently bound to the front surface and a first nucleotide, as provided by a method of the present invention and as further described below. The Link serves to functionalize the surface for binding by the first nucleotide during the in situ process. “Cap” represents a capping agent which may or may not be present. The Link may be any of the “second silanes” referenced in U.S. Pat. No. 6,444,268 while the Cap may be any of the “first silanes” in that patent. However, different linking layer compositions than those silanes could be used. As already mentioned, the foregoing patents are incorporated herein by reference, including for example the details of the linking layer compositions used therein.

Substrate 10 has one or more identifiers in the form of bar codes 356. Identifiers such as other optical or magnetic identifiers could be used instead of bar codes 356 which will carry the information discussed below. Each identifier may be associated with its corresponding array by being positioned adjacent that array 12. However, this need not be the case and identifiers such as bar code 356 can be positioned elsewhere on substrate 10 if some other means of associating each bar code 356 with its corresponding array is provided (for example, by relative physical locations). Further, a single identifier might be provided which is associated with more than one array 12 on a same substrate 10 and such one or more identifiers may be positioned on a leading or trailing end of substrate 10. The substrate may further have one or more fiducial marks 18 for alignment purposes during array fabrication.

Arrays 12 may be fabricated on the functionalized surface 11a by depositing onto the continuous functionalized area on the substrate surface, drops containing the probe precursors and viscosity modifier, as described above, at the multiple feature locations of the array to be fabricated, so that the probes or probe precursors bind to the linking agent at the feature locations. This step is repeated in subsequent “cycles” at one or more features. Such methods and their chemistry are described in detail in the references cited in the “Background” section above. At least one additional step may occur between each cycle, such as oxidation of a phosphite bond to phosphate and deprotection of the 5′ (or 3′ in a reverse synthesis) hydroxy of a nucleoside phosphoramidite deposited and linked in the preceding cycle.

FIGS. 2 and 3 illustrate features 16 of an array 12 in more detail, where the actual features formed are the same as target (or “aim”) features, with each feature 16 being uniform in shape, size and composition, and the features being regularly spaced. Conventionally features 16 would be perfectly circular in shape. To form perfectly an array with perfectly circular features by drop deposition methods would require all reagent droplets for each feature to be uniform in shape and all accurately deposited at the target feature location. However, in arrays of the present invention, as shown, features 16 need not be circular and may even be slightly oblong in shape.

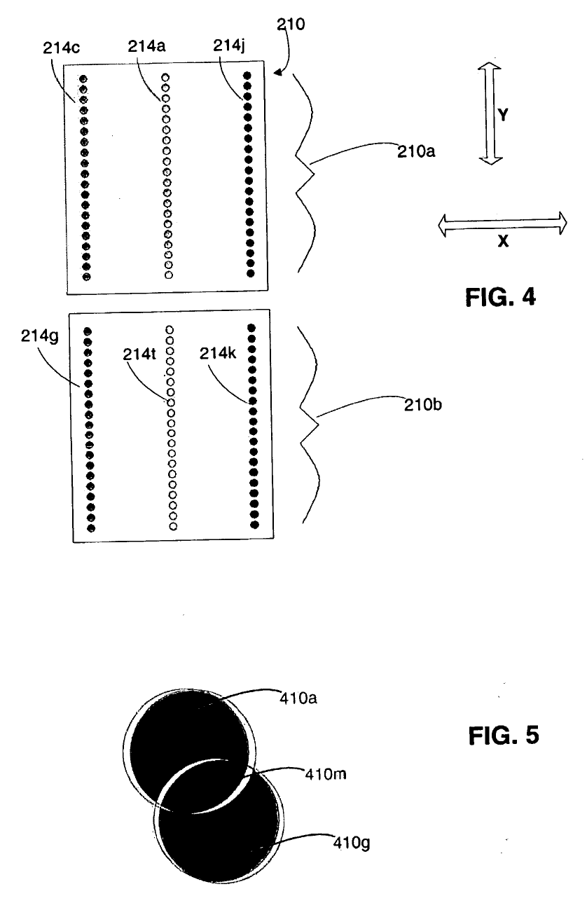

Operation of particular embodiments of methods of the present invention can be understood with reference to FIGS. 4-10. While probe precursors in the form of nucleoside phosphoramidite monomers will particularly be referenced in much of the following description, it will be appreciated that other probe precursors (for example, amino acids) can be used instead, in accordance with the methods described in this application. Also, while one drop of a particular reagent may be referenced it will be understood that this can be replaced, for example, by many smaller drops from the a same nozzle.

Referring in particular to FIG. 4 a head system 210 of an apparatus used to fabricate arrays according to a method of the present invention includes a set of two heads 210a, 210b. As pointed out below heads 210a, 210b are mounted in a head retainer 208 for simultaneous movement together as a unit with respect to a substrate 10 on which an array is to be fabricated. Note that throughout this application this “movement” of heads 210a, 210b is relative with respect to the substrate 10, and either the head system 210 or substrate 10 may actually be moved while the other is held stationary, or they may both be moved. In practice it may be particularly convenient that the head system 210 is in fact stationary during array fabrication while the substrate 10 is moved beneath head system 210. However, in this case the heads 210a, 210b may still be moved relative to one other for alignment purposes.

Head 210a has three rows of nozzles 214c, 214a, 214j through which drops of reagents are dispensed containing either one of the four phosphoramidites or a tetrazole activator. In particular, nozzles 214c dispense C, nozzles 214a dispense A, and nozzles 214j dispense tetrazole activator. Similarly, on head 210b nozzles 214g dispense G, nozzles 214t dispense T, and nozzles 214k dispense tetrazole. In operation head system 210 will move relative to a substrate 10 in a direction left and right as viewed in FIG. 4 (“X” direction). Orifices within a given head are fixed in position relative to one another by being fabricated in a one-piece orifice plate member of the head. In a prior art method when one or more drops of a nucleoside phosphoramidite were deposited from a head onto a feature location during a cycle, during that same cycle one or more drops of activator would also be deposited onto the feature location from the same head. For example, if a drop of G phosphoramidite was dispensed from nozzle 214g of head 210b onto a feature during a cycle, then during that same cycle the activator would be dispensed in that prior art method onto that same feature from a nozzle 214k (typically nozzles 214k and 214g which each dispense a drop to a same feature during a same cycle, will lie along the same line in the X direction).

The present invention recognizes that in the situation where heads 210a and 210b are perfectly aligned the foregoing prior art method works fine. By different heads being “aligned” in this context is meant that when the head system is repositioned in the X and/or Y direction such that a nozzle at a given position within one head will be exactly functionally superimposed over a nozzle at the like position within the other head, other nozzles (ideally all such other nozzles) in the other head will simultaneously be exactly functionally superimposed over other nozzles of the one head. “Functional superimposition” of “nozzles” in this context is measured based on drops being deposited from the nozzles to a fixed distance surface being coextensive, with all factors other than X, Y relative positioning of the head and surface remaining the same. For example, in FIG. 4 when the uppermost nozzle in row 214a is functionally superimposed over uppermost nozzle in row 214t, each of the other nozzles in head 210a are simultaneously functionally superimposed over nozzles in like locations within head 210b. Heads 210a, 210b can be out of alignment due to an error in any of their expected X, Y or Z positioning relative to each other, or one head being tilted at an angle relative to the other in any of the XY, XZ, or YZ planes. While functional alignment or mis-alignment will normally be the result of physical alignment or mis-alignment of the heads in space (such as due to the foregoing errors or tilting) it will be appreciated that such could also result from other non-physical errors such as timing errors in firing nozzles. With this understanding it will be further appreciated that when a functional mis-alignment is actually desired (as described below in connection with FIG. 10) such can be obtained using means other than physical mis-alignment of the heads in space (for example, by timing adjustment even when the heads are physically aligned).

However, when different heads such as heads 210a, 210b are out of alignment the foregoing prior art method can result in cycle to cycle errors in array features 16 which contain unexpected and undesired biopolymer sequences. This can be understood with reference to FIG. 5 in particular. In FIG. 5 a drop of A has been deposited from a nozzle 214a in head 210a for a feature location along with a drop of activator from a nozzle 214j from the same head 210a during the same cycle (for simplicity it will be assumed this is the first cycle of in situ synthesis). Following oxidation, washing, deprotection and drying procedures prior to the next cycle, the result is a region 410a carrying only dried probe precursor (that is, A) linked to the substrate surface. In a further (in this example, next) cycle G is deposited from head 210b as well as a drop of tetrazole, for the same feature location. However, head 210b is mis-aligned with respect to head 210a. As a result, G will link only in region 410g, and only in the region of overlap at 410m will the desired sequence AG be fabricated. The non-overlapping portion of region 410a contains A only and the non-overlapping portion of region 410g contains G only. If further cycles are executed (with intermediate process steps, including drying) it can be seen that only the overlapping portion at 410m will contain the desired sequence to be synthesized while the non-overlapping portions of regions 410a, 410g will contain undesired shorter sequences.

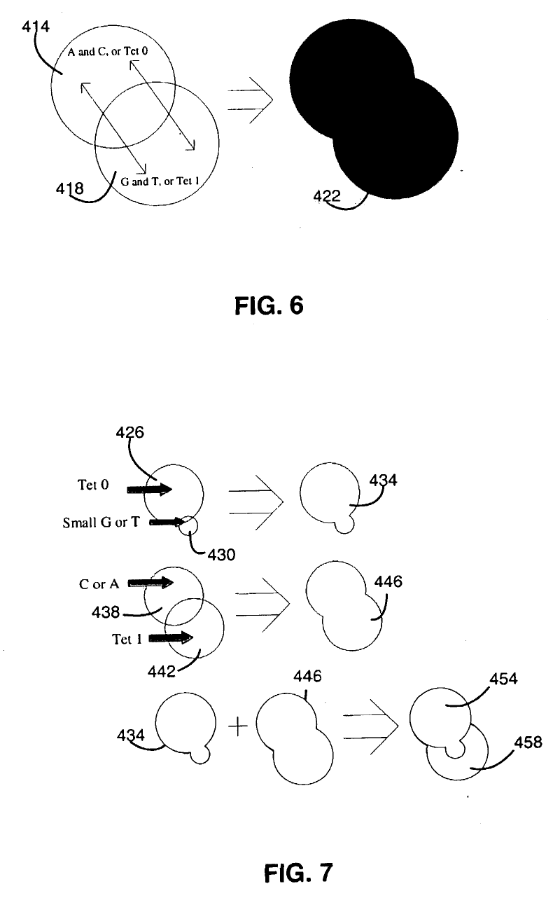

The present invention recognizes that the situation illustrated in FIG. 5 can be reduced or avoided if, for each cycle, rather than depositing drops for a feature from a same head of a set of heads during a same cycle, they are instead deposited from different heads of the set. That is, drops are deposited at a same feature from the set of heads during a one cycle, and further drops are deposited at the same feature from the set of heads during another cycle. For example, if during one cycle a drop of phosphoramidite A or C is deposited from head 210a for a feature (and covers region 414 in FIG. 6) and a drop of tetrazole activator is deposited from head 210b for the same feature in the same cycle (and covers region 418), and during another cycle a drop of G or T is deposited for the same feature from head 210b (covering region 418) and a drop of tetrazole activator is deposited from head 210a (covering region 414), the resulting feature carrying the desired sequence will cover the region 422 in FIG. 6. Note that region 422 is the union of regions 414,418 rather than an intersection as was the case in FIG. 4. Also note that in FIG. 5 there are no regions carrying undesired sequences shorter than the desired full-length sequence.

The advantageous situation illustrated in FIG. 5 assumes that that all drops deposited at the same feature location from a same head during a same cycle, together have an area of coverage which is substantially constant across different heads and different cycles. In particular, in FIG. 4 it is assumed that the area of regions 414 and 418 are substantially the same. In some situations it is possible that this may not be the case (for example, where the G or T dispensing nozzles in head 210b consistently cover a smaller area on the surface than: the tetrazole dispensing nozzles 214k of head 210b; and the A and C dispensing nozzles of head 210a). Even in this situation though, the advantage of the present method can still be realized when all the drops deposited at the same feature location from the different heads during a same cycle together cover an area on the surface which is substantially coextensive over different cycles. This can be understood with reference to FIGS. 7 and 8.

The first row of FIG. 7 illustrates a deposited drop of G or T from head 210b covering region 430 while a deposited drop of tetrazole from head 210a for the same feature location during the same cycle would cover region 426. Since the drops are present during the same cycle they merge to form region 434. After intermediate processing steps between cycles as described previously, region 434 will represent only G or T linked to surface 11a. In a next cycle a C or A is deposited from head 210a and covers region 438 while a tetrazole drop deposited from head 210b covers region 442, as illustrated in the second row of FIG. 7. Note that in the second row of FIG. 7 the region 430 is not shown for clarity. Also, the region 430 is much smaller in area than the other regions 426, 438, 442 (the volume of G or T deposited from nozzles was less than A or C or tetrazole from other nozzles). After the intermediate processing steps C or A is left linked over a region 446. However, the overall result of the two cycles is shown in the third row of FIG. 7 where the region 454 (of the same shape as region 434) represents the region of desired polynucleotide sequence while region 458 represent the region of undesired truncated sequence Oust C or A).

However, from FIG. 7 it will be appreciated that undesired region 458 can be avoided even in the event of smaller drops forming smaller regions such as region 430. In particular, if all the drops deposited at the same feature location from the different heads during a same cycle together cover an area on the surface which is substantially coextensive over different cycles. That is, regions such as 434 and 446 would be coextensive although they would have to be formed differently than in the first row of FIG. 7 One way of accomplishing this desired result is to reduce the area of region 442 to match area 430 by reducing the volume of tetrazole from head 210b during the second cycle. Another way is to ensure that all the drops deposited at the same feature location from a same head during a same cycle together have an area of coverage which is substantially constant across different heads and different cycles (this is the situation in FIG. 6). A still further way of obtaining the foregoing desired result is illustrated in FIG. 8.

In FIG. 8, in one cycle the drops deposited at the same feature from the set of heads are a drop of a G or T probe precursor from head 210b (occupying region 430 on the substrate surface), a drop of a tetrazole activator from the head 210b (occupying region 426), and also a drop of tetrazole activator from head 210a (occupying region 432 in FIG. 8). This would result in a linked region 434 after the usual intermediate processing steps. If the second cycle of FIG. 7 were then executed at this feature location the final region of the desired biopolymer would still have the shape of region 434 with no undesired truncated sequence regions (such as region 458). In practice, one may not always know in advance if at a given feature a drop of probe precursor will be unexpectedly small thereby leading to the situation of FIG. 7 over multiple cycles, at that feature. In this case, the result of FIG. 7 can still be avoided by repeating the process of FIG. 8 in each of the cycles. That is, when in another cycle a drop of probe precursor is deposited at the same feature as in FIG. 8 from head 210a, then a drop of the tetrazole activator is also deposited from head 210a and a drop of tetrazole activator deposited from head 210b. If the coverage area of deposited drops of activator from both heads in each of multiple cycles, is selected to always be substantially greater than the coverage area of a deposited probe precursor drop during a same cycle, then the resulting feature can have a shape such as region 434 with no regions of undesired truncated sequences.

The method illustrated in FIG. 8 can also be used to adjust an aspect ratio (that is, a ratio of length to width) of features of a fabricated array. This can be understood with reference to FIGS. 9 and 10. In particular, in FIG. 9 the drops deposited at the same feature from the set of heads 210a, 210b during one cycle consist of a series of drops 450a, 450b, 450c of a probe precursor (G or T) from a same nozzle of first head (head 210b in this example) and a series of drops 458a, 458b, 458c of the activator deposited from the same nozzle of a second head (head 210a). Note that the drops within each series are displaced relative to one another along a first direction (X in FIGS. 9 and 10). Assuming heads 210a, 210b are aligned then following intermediate processing the linked probe will occupy an oblong region 470 in FIG. 9. If it is desired to reduce the aspect ratio of shape 470 (that is, decrease the length/width ratio) this can be accomplished by purposely mis-aligning heads 210a, 210b so that now the series of drops from different heads at a feature during a same cycle (in particular, the series 454 and 458) are displaced relative to one another in a direction (Y in FIG. 10) crosswise to the direction X. This same procedure would be repeated during each of the other cycles although in another cycle the series of drops of different probe precursor may be from the second head (head 210a) and a series of drops of the activator from the first head (head 210b). Such reduced aspect ratio features can aid in automatic location of features during array reading by software which expects to find approximately circular features.

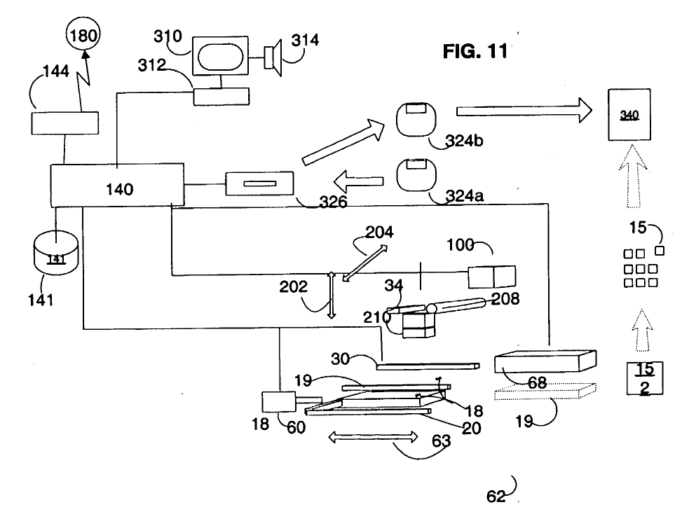

Referring now to FIG. 11, an apparatus of the present invention that can execute a method of the present invention, is illustrated. This apparatus is configured for use with a large substrate 19 which will later be cut into individual substrates 10 of any of the array assemblies 15. Substrate 19 will therefore also be referred to as having surfaces 11a and 11b. The apparatus of FIG. 11 includes substrate station 20 (sometimes referenced as a “substrate holder”) on which a substrate 19 can be mounted and retained. Pins or similar means (not shown) can be provided on substrate station 20 by which to approximately align substrate 19 to a nominal position thereon (with alignment marks 18 on substrate 19 being used for more refined alignment). Substrate station 20 can include a vacuum chuck connected to a suitable vacuum source (not shown) to retain a substrate 19 without exerting too much pressure thereon, since substrate 19 is often made of glass. A flood station 68 is provided which can expose the entire surface of substrate 19, when positioned at station 68 as illustrated in broken lines in FIG. 11, to a fluid typically used in the in situ process, and to which all features must be exposed during each cycle (for example, oxidizer, deprotection agent, and wash buffer). In the case of deposition of a previously obtained polynucleotide, flood station 68 need not be present.

A drop deposition system is present in the form of a dispensing head system 210 which is retained by a head retainer 208. The head system includes heads 210a, 210b retained by the same head retainer 208 so that such retained heads move in unison together. The transporter system includes a carriage 62 connected to a first transporter 60 controlled by processor 140 through line 66, and a second transporter 100 controlled by processor 140 through line 106. Transporter 60 and carriage 62 are used execute one axis positioning of station 20 (and hence mounted substrate 19) facing head system 210, by moving it in the direction of axis 63 (X direction). Transporter 100 provide adjustment of the position of head retainer 208 (and hence head system 210) in a direction of axis 204 and therefore move head system 210 in the direction of travel 204a which is one direction on axis 204 (Y direction). In this manner, head system 210 can be scanned line by line along parallel lines in a raster fashion, by scanning along a line over substrate 19 in the direction of axis 204 using transporter 100, while line to line transitioning movement of substrate 19 in a direction of axis 63 is provided by transporter 60. Transporter 60 can also move substrate holder 20 to position substrate 19 in station 68 (as illustrated by the substrate 19 shown in broken lines in FIG. 11) at which the intermediate processing steps are completed. Head system 210 may also optionally be moved in a vertical direction 202, by another suitable transporter (not shown) and its angle of rotation with respect to head system 210 also adjusted. It will be appreciated that other scanning configurations could be used during array fabrication. It will also be appreciated that both transporters 60 and 100, or either one of them, with suitable construction, could be used to perform the foregoing scanning of head 210 with respect to substrate 19. Thus, when the present application recites “positioning”, “moving”, or similar, one element (such as head 210) in relation to another element (such as one of the stations 20 or substrate 19) it will be understood that any required moving can be accomplished by moving either element or a combination of both of them. Head system 210, the transporter system, and processor 140 together act as the deposition system of the apparatus. An encoder 30 communicates with processor 140 to provide data on the exact location of substrate station 20 (and hence substrate 19 if positioned correctly on substrate station 20), while encoder 34 provides data on the exact location of holder 208 (and hence head system 210 if positioned correctly on holder 208). Any suitable encoder, such as an optical encoder, may be used which provides data on linear position.

Holder 208 includes a mechanical adjustment mechanism which allows for adjustment of the physical alignment of heads 210a, 210b with respect to one another in space. One particularly useful mechanism in holder 208 for obtaining such adjustment, is disclosed in U.S. patent application Ser. No. 10/022065 titled “Multiple Axis Printhead Adjuster For Non-Contact Fluid Deposition Devices” and filed Dec. 18, 2001. As mentioned above, the foregoing application, and particularly the details of the adjusting mechanism, are incorporated herein by reference.

Processor 140 also has access through a communication module 144 to a communication channel 180 to communicate with a remote station. Communication channel 180 may, for example, be a Wide Area Network (“WAN”), telephone network, satellite network, or any other suitable communication channel.

Each of one or more heads 210a may be of a type similar to that used in an ink jet type of printer and may, for example, include three or more chambers (at least one for each of two nucleoside phosphoramidite monomers plus at least one for an activator solution) each communicating with a corresponding set of multiple drop dispensing orifices and multiple ejectors which are positioned in the chambers opposite respective orifices. The orifices of a same head 210a or 210b are in a one-piece (that is, unitary) orifice plate. Each ejector is in the form of an electrical resistor operating as a heating element under control of processor 140 (although piezoelectric elements could be used instead). Each orifice with its associated ejector and portion of the chamber, defines a corresponding pulse jet. It will be appreciated that each head 210a, 210b could, for example, have more or less pulse jets as desired (for example, at least ten or at least one hundred pulse jets, with their nozzles organized in rows and columns). Application of a single electric pulse to an ejector will cause a droplet to be dispensed from a corresponding orifice. Certain elements of the head 210 can be adapted from parts of commercially available piezoelectric inkjet heads. One type of suitable head construction is described in U.S. Pat. No. 6,461,812, incorporated herein by reference. However, each head 210 could instead dispense only one probe precursor (for example, just one of the four nucleoside phosphoramidites) as well as an activator. The number of heads would be correspondingly increased and the number of chambers in each could be correspondingly decreased. Other multiple head configurations will be apparent.

The amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating or piezoelectric element, as well as the material properties of the liquid being dispensed (such as viscosity and surface tension). The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As discussed above, when the orifice is in motion with respect to the substrate surface at the time an ejector is activated, the actual site of deposition of the material will not be the location that is at the moment of activation perpendicularly aligned with an orifice. However, the actual deposited location will be predictable for the given distances and velocities. As previously mentioned, a series of drops of the same composition can be dispensed from a same pulse jet to a same feature location during a same cycle, rather than one larger drop of volume equivalent to the total volume of the series.

The apparatus further includes a display 310, speaker 314, and operator input device 312. Operator input device 312 may, for example, be a keyboard, mouse, or the like. Processor 140 has access to a memory 141, and controls print head system 78 and print head 210 (specifically, the activation of the ejectors therein), operation of the transporter system and the third transporter 72, and operation of display 310 and speaker 314. Memory 141 may be any suitable device in which processor 140 can store and retrieve data, such as magnetic, optical, or solid state storage devices (including magnetic or optical disks or tape or RAM, or any other suitable device, either fixed or portable). Processor 140 may include a general purpose digital microprocessor suitably programmed from a computer readable medium carrying necessary program code, to execute all of the steps required by the present invention, or any hardware or software combination which will perform those or equivalent steps. The programming can be provided remotely to processor 141 through communication channel 180, or previously saved in a computer program product such as memory 141 or some other portable or fixed computer readable storage medium using any of those devices mentioned below in connection with memory 141. For example, a magnetic or optical disk 324a may carry the programming, and can be read by disk writer/reader 326. A cutter 152 is provided to cut substrate 19 into individual array assemblies 15.

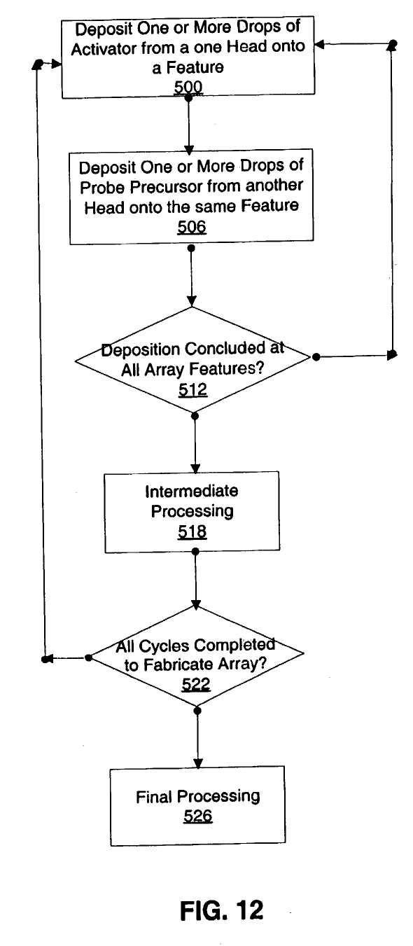

The operation of the apparatus of FIG. 11 will now be described. In the following description reference numbers in parentheses refer to the flowchart of FIG. 12. It will be assumed that a substrate with a functionalized surface 11a is provided on substrate station 20 either manually or by a robot arm (not shown). It will be assumed that processor 140 is programmed with the necessary layout information to fabricate target arrays 12 using any of the methods (including drop splat diameters) discussed above. Such information on the layout would have already taken into account the splat dimensions of the drops to be deposited, in the manner described above. Using information such as the foregoing target layout and the number and location of drop dispensers in head 210, processor 140 can then determine a reagent drop deposition pattern. Alternatively, such a pattern could have been determined by another processor (such as a remote processor) and communicated to memory 141 through communication channel 180 or by forwarding a portable storage medium carrying such pattern data for reading by reader/writer 326. Processor 140 controls fabrication, in accordance with the deposition pattern, to generate the one or more arrays 12 on each section of substrate 19 which will later be cut into each substrate 10. This is done by depositing (500) one or more drops of an activator from one head onto a feature location. One or more drops of probe precursor are deposited (506) from another head onto the same feature. The foregoing events (500, 506) may be reversed in order or occur simultaneously. Also, these will typically occur for multiple features at the same time given the construction of heads 210a, 210b. If it is determined (512) that deposition has not yet concluded for some features the foregoing (500, 506) is process is executed at those features until it is determined (512) that drops have been deposited for all array features. This repetition at further features occurs by scanning the head system 210 in relation to the substrate surface 11a in a row by row raster fashion. No drops are dispensed for features or otherwise during row transitioning. The probe or probe precursors will bind to the different regions through the linker agent. At this point a cycle is complete and the substrate and processor 140 then sends substrate 19 to station 68 for cycle intermediate processing (518), all in accordance with the conventional in situ polynucleotide array fabrication process described above. Processor 140 then determines (522) if all cycles required to fabricate the array were completed. If not, the foregoing steps are repeated in each of multiple cycles with intermediate processing between each cycle. If all cycles are completed then substrate 19 is sent to a chamber (not shown) for final ammonia deprotection in a manner well known in in situ synthesis chemistry. During any cycle at any feature, details of drop deposition can be executed in accordance with those methods described above.

Processor 140 then sends substrate 19 to a cutter 152 wherein sections of substrate 19 are separated into substrates 10 carrying one ore more arrays 12, to provide multiple array assemblies 15. One or more array assemblies 15 may then be forwarded to one or more remote users. The foregoing array fabrication sequence can be repeated at the fabrication station as desired for multiple substrates 19 in turn.

During array fabrication errors can be monitored and used in any of the manners described in U.S. Patent Application “Polynucleotide Array Fabrication” by Caren et al., Serial No. 09/302898 filed Apr. 30/99, and U.S. Pat. No. 6,232,072. Also, the one or more identifiers in the form of bar codes 356 can be attached or printed onto sections of substrate 19 defining the substrates 10 before entering, or after leaving, first fabrication station 70, or after leaving the second fabrication station 20. Regardless of the foregoing, at any point in the operation of the apparatus of FIG. 11, processor 140 will associate each array with an identifier such as a bar code 356, which identifier carries an indication of the array layout and any other desired information regarding the array or its fabrication parameters, or is linked to a file carrying such information. The file and linkage can be stored by processor 140 and saved into memory 141 or can be written onto a portable storage medium 324b which is then placed in the same package 340 as the corresponding array assembly 15 for shipping to a remote customer. Optionally other characteristics of the fabricated arrays can be included in the code 356 applied to the array substrate or a housing, or a file linkable to such code, in a manner as described in the foregoing patent application and U.S. Pat. No. 6,180,351. As mentioned above, this reference is incorporated herein by reference.

Suitable apparatus and in situ methods for fabricating arrays 12 are further described in U.S. Pat. No. 6,180,351, U.S. Pat. No. 6,242,266, U.S. Pat. No. 6,306,599, and U.S. Pat. No. 6,420,180. As mentioned above, the foregoing references are incorporated herein by reference particularly as relates to the in situ fabrication apparatus and methods disclosed therein.

Following receipt by a user of an array made according to the present invention, the array will typically be exposed to a sample (for example, a fluorescently labeled polynucleotide or protein containing sample) and the array then read. All arrays 12 on substrate 10 can be read at the same time or not by using any suitable reading apparatus. Where fluorescent light is to be detected due to incorporation of fluorescent labels into the target in a known manner, well known array readers can be used. For example, such a reader may scan one or more illuminating laser beams across each array in raster fashion and any resulting fluorescent signals detected, such as described in U.S. Pat. No. 6,406,849. One such scanner that may be used for this purpose is the AGILENT MICROARRAY SCANNER manufactured by Agilent Technologies, Palo Alto, CA. However, arrays may be read by any other method or apparatus than the foregoing, with other reading methods including other optical techniques (for example, detecting chemiluminescent or electroluminescent labels) or electrical techniques (where each array feature is provided with an electrode to detect hybridization at that feature in a manner disclosed in U.S. Pat. No. 6,251,685, U.S. Pat. No. 6,221,583 and elsewhere).

Results from the array reading can be further processed results, such as obtained by rejecting a reading for a feature which is below a predetermined threshold and/or forming conclusions based on the pattern read from the array (such as whether or not a particular target sequence may have been present in the sample or an organism from which the sample was obtained exhibits a particular condition or disease). The results of the reading (processed or not) can be forwarded (such as by communication) to be received at a remote location for further evaluation and/or processing, or use, using communication channel 180 or reader/writer 186 and medium 190. This data may be transmitted by others as required to reach the remote location, or re-transmitted to elsewhere as desired.

Note that nozzles within a head (particularly one in which the nozzles are formed in a one-piece orifice plate) are usually well aligned, but when multiple heads are used it is difficult to ensure the different heads are in alignment. Methods of the present invention can reduce the need for accurate head alignment.

Various other modifications to the particular embodiments described above are, of course, possible. Accordingly, the present invention is not limited to the particular embodiments described in detail above.

What is claimed is:

1. A method of fabricating an array of biopolymer probes bound to a surface of a substrate at feature locations, using deposition heads each comprising a set of fixed nozzles through which drops are dispensed, the method comprising for each of multiple feature locations:
(a) depositing drops during a same cycle onto the feature location from a set of the deposition heads while spaced from the surface, at least some of which drops contain probe precursors, so that the probe precursors bind to the surface through a linker;
(b) repeating (a) multiple times wherein the probe precursor deposited in a prior cycle becomes the linker for a probe precursor deposited in a subsequent cycle;
wherein drops are deposited at a same feature from the set of heads during a one cycle, and further drops are deposited at the same feature from the set of heads during another cycle.

2. A method according to claim 1 wherein the biopolymer probes comprise polynucleotide or peptide probes.

3. A method according to claim 2 wherein:
the set of heads comprises a first head and a second head;
the drops deposited at the same feature from the set of heads during the one cycle comprise a drop of a probe precursor from the first head and a drop of an activator from the second head; and
the drops deposited at the same feature in the other cycle comprise a drop of a probe precursor from the second head and a drop of the activator from the first head.

4. A method according to claim 3 wherein the probe precursor deposited at the same feature in the one cycle is different from the probe precursor deposited during the other cycle.

5. A method according to claim 2 wherein:
the set of heads comprises a first head and a second head;
the drops deposited at the same feature from the set of heads during the one cycle comprise a series of drops of the probe precursor from the first head and a series of drops of an activator from the second head; and
the drops deposited at the same feature in the other cycle comprise a series of drops of the probe precursor from the second head and a series of drops of the activator from the first head.

6. A method according to claim 3 wherein all the drops deposited at the same feature location from the different heads during a same cycle together cover an area on the surface which is substantially coextensive over different cycles.

7. A method according to claim 6 wherein all the drops deposited at the same feature location from a same head during a same cycle together have an area of coverage which is substantially constant across different heads and different cycles.

8. A method according to claim 6 wherein:
the set of heads comprises a first head and a second head;
the drops deposited at the same feature from the set of heads during a same cycle comprise a drop of a probe precursor from the first head and a drop of an activator from the first head and from the second head.

9. A method according to claim 6 wherein:
the set of heads comprises a first head and a second head;
the drops deposited at the same feature from the set of heads during the one cycle comprise a drop of a probe precursor from the first head and a drop of an activator from the first head and from the second head; and
the drops deposited at the same feature in the other cycle comprise a drop of a probe precursor from the second head and a drop of the activator from the first head and from the second head.

10. A method according to claim 9 wherein the activator deposited from the first and second heads at the same feature during each of the cycles has a coverage area which is greater than the probe precursor deposited during the same cycle.

11. A method according to claim 5 wherein the drops within each series at the same feature are displaced relative to one another along a first direction while series from different heads are displaced relative to one another in a direction crosswise to the first direction.

12. A method according to claim 6 wherein drops of an activator and probe precursor are dispensed from the first and second heads, with different probe precursors being dispensed from the first and second heads.

13. A method according to claim 12 wherein the biopolymer probe comprises polynucleotides and wherein an activator and a pair of nucleoside monomers are dispensed from each of the first and second heads, with different pairs of nucleoside monomers being dispensed from the first and second heads.

14. A method according to claim 12 wherein the same activator is dispensed from the different heads.

15. A method according to claim 6 additionally comprising exposing the fabricated array to a sample.

16. A method according to claim 15 additionally comprising, following exposure of the array to a sample, reading the array.

17. A method comprising forwarding a result of a reading obtained by the method of claim 16, to a remote location.

18. A method comprising receiving a result of reading obtained by the method of claim 16 from a remote location.

19. A method according to claim 1 wherein the probes comprise polynucleotide probes and the probe precursors comprise nucleoside phosphoramidites.

20. A method according to claim 3 wherein the probes comprise polynucleotide probes and the probe precursors comprise nucleoside phosphoramidites.

21. A method according to claim 6 wherein the probes comprise polynucleotide probes and the probe precursors comprise nucleoside phosphoramidites.

22. A method according to claim 1 wherein the set of nozzles of a head are formed in a one-piece member.

23. A computer program product, comprising: a computer readable storage medium having a computer program stored thereon which executes a method of claim 1 when loaded into a computer.

24. A computer program product, comprising: a computer readable storage medium having a computer program stored thereon which executes a method of claim 3 when loaded into a computer.

25. A computer program product, comprising: a computer readable storage medium having a computer program stored thereon which executes a method of claim 6 when loaded into a computer.

26. A computer program product, comprising: a computer readable storage medium having a computer program stored thereon which executes a method of claim 8 when loaded into a computer.

27. An array fabrication apparatus comprising:
multiple deposition heads each comprising multiple fixed nozzles through which drops are dispensed;
a processor which controls operation of the deposition heads so as to execute a method of claim 1.

28. An array fabrication apparatus comprising:
multiple deposition heads each comprising multiple fixed nozzles through which drops are dispensed;
a processor which controls operation of the deposition heads so as to execute a method of claim 3.

29. An array fabrication apparatus comprising:
multiple deposition heads each comprising multiple fixed nozzles through which drops are dispensed;
a processor which controls operation of the deposition heads so as to execute a method of claim 6.

30. An array fabrication apparatus comprising:
multiple deposition heads each comprising multiple fixed nozzles through which drops are dispensed;
a processor which controls operation of the deposition heads so as to execute a method of claim 8.

31. A method according to claim 30 wherein the set of nozzles of a head are formed in a one-piece member.

32. A method of fabricating an array of biopolymer probes bound to a surface of a substrate at feature locations, using a set of deposition heads each comprising a set of nozzles through which drops are dispensed, the method comprising:
(a) adjusting the relative orientation of heads in the set so as to simultaneously adjust the trajectories of drops ejected from a set of nozzles of one head relative to a set of nozzles of another head;
(b) fabricating an array comprising, for each of multiple feature locations:
(i) depositing drops during a same cycle onto the feature location from a set of the deposition heads while spaced from the surface, at least some of which drops contain probe precursors, so that the probe precursors bind to the surface through a linker;
(ii) repeating (i) multiple times wherein the probe precursor deposited in a prior cycle becomes the linker for a probe precursor deposited in a subsequent cycle;
wherein drops are deposited at a same feature from the set of heads during a one cycle, and further drops are deposited at the same feature from the set of heads during another cycle.

33. A method according to claim 32 wherein the probes comprise polynucleotide probes.

34. A method according to claim 33 wherein:
the set of heads comprises a first head and a second head;
the drops deposited at the same feature from the set of heads during the one cycle comprise a drop of a probe precursor from the first head and a drop of an activator from the second head; and
the drops deposited at the same feature in the other cycle comprise a drop of a probe precursor from the second head and a drop of the activator from the first head.

35. A method according to claim 34 wherein the probe precursor deposited at the same feature in the one cycle is different from the probe precursor deposited during the other cycle.

36. A method according to claim 32 wherein the drops deposited at the same feature location from the different heads during a cycle together cover an area on the surface which is substantially coextensive across different cycles.

37. A method according to claim 1 wherein the set of nozzles of a head are formed in a one-piece member.
